SEQUENCE LISTING (8 sequences)

SEQ ID NO: 1 - protein, 10 aa, Artificial Sequence
  HIS6G4
HHHHHHGGGG

SEQ ID NO: 2 - protein, 8 aa, Artificial Sequence
  G2H6
GGHHHHHH

SEQ ID NO: 3 - protein, 20 aa, Artificial Sequence
  150F Primer
GCTGGAGTGAGTGGGAAGAG

SEQ ID NO: 4 - protein, 20 aa, Artificial Sequence
  150R Primer
GCTGGAGTGAGTGGGAAGAG

SEQ ID NO: 5 - protein, 20 aa, Artificial Sequence
  500F primer
ACAGCACGGAACGGGTGAAG

SEQ ID NO: 6 - protein, 20 aa, Artificial Sequence
  500R primer
ACAGCACGGAACGGGTGAAG

SEQ ID NO: 7 - protein, 20 aa, Artificial Sequence
  850F primer
CCAGCTGTCTGCACAGGAGA

SEQ ID NO: 8 - protein, 20 aa, Artificial Sequence
  850R primer
CCAGCTGTCTGCACAGGAGA

(19) United States
(12) Patent Application Publication
(10) Pub. No.: US 2016/0318982 A1
(43) Pub. Date: Nov. 3, 2016

(54) METHOD FOR PREPARING SINGLE-VALENT, MODIFIED AVIDIN-LIKE MOLECULES AND MODIFIED FUNCTIONAL CONJUGATES

(71) Applicant: CHUNG YUAN CHRISTIAN UNIVERSITY, TAOYUAN CITY (TW)

(72) Inventors: Cheng-An LIN, New Taipei City (TW); Ting-En LIN, Taipei City (TW); Wei-Ling HUNG, New Taipei City (TW); Wei-Xu CHEN, New Taipei City (TW)

(21) Appl. No.: 14/699,671

(22) Filed: Apr. 29, 2015

(51) Int. Cl. C07K 14/465; C07K 1/13; C07K 1/22

(52) U.S. Cl. CPC C07K 14/465; C07K 1/22; C07K 1/13

(57) ABSTRACT

Disclosed herein are methods for preparing modified, functional conjugates that includes the steps as follows. First, biotinylated peptides, each having a His-tag sequence, are gradually added into avidin-like molecules in a molar ratio between 1:4 to 16:1 so as to form a plurality of avidin-biotin-peptide conjugates with different valence states. The avidin-biotin-peptide conjugates are then separated using a nickel affinity column eluted with an imidazole gradient to produce a first eluate including mostly first single-valent avidin-biotin-peptide conjugates each having a first valence. The first single-valent avidin-biotin-peptide conjugates are then mixed with functional molecules under conditions allowing the formation of functional molecules modified with the first single-valent biotin-binding peptides of the first valence. Methods for preparing single-valent, modified avidin-like molecules are also disclosed herein.

### BACKGROUND OF THE INVENTION

1. Field of the Invention

The present disclosure relates to methods for preparing modified functional conjugates. More particularly, the disclosed invention relates to methods for preparing modified functional conjugates using single-valent, grafted avidin-like molecules.

2. Description of Related Art

In the biomedical field, molecules with biological and/or pharmaceutical functions have been widely used as research tools or in disease diagnosis and/or treatment. Examples of such functional molecules include, but are not limited to, nucleotides, peptides, antibodies, antigens, nanoparticles, quantum dots, fluorescent materials, magnetic materials, and therapeutics.

With the advancement and progress of material science, functional molecules are often modified to attain additional properties. For examples, in biomedical fields, nanoparticles are often surface-modified with cell-targeting or cell-penetrating moieties to enhance the in vivo delivery of the nanoparticles. Alternatively, the surface of nanoparticles can be grafted with bioactive moieties (e.g., nucleotides, proteins, and antibodies) or therapeutics to impart biologically active properties thereto. Moreover, it is desirable to modify biomedical probes, such as DNA probes, antibody probes, fluorescent probes, and folic acid probes, with suitable moieties to increase the labeling efficacy or biocompatibility thereof.

The number of moieties grafted onto a functional molecule may affect the properties of the resultant modified functional molecule. For instance, Howarth et al. established that mono-valent Alexa Fluor 568-conjugated streptavidin grafted with only one biotinylated AP-neuroligin-1 moiety exhibited better imaging resolution against hippocampal neurons, as compared with tetra-valent Alexa Fluor 568-conjugated streptavidin grafted with four biotinylated AP-neuroligin-1 moieties did (see, Howarth et al. A monovalent streptavidin with a single femtomolar biotin binding site. Nature Methods. Vol. 3, p. 267-273, 2006).

Also, the grafted moieties may affect the three-dimensional structure of the modified functional molecules. In the case where the functional molecules are to be assembled into nanoparticles, the attachment of multiple moieties to a functional molecule may hinder the assembling process.

In view of the foregoing, it is important to control the number of moieties grafted onto each functional molecule precisely. However, most methods for modifying functional molecules give a mixture containing modified functional molecules with different numbers of moieties grafted therewith, and it is difficult to quantify the moieties comprised in the mixture, which may be problematic in many aspects. For biomedical probes grafted with cell-targeting moieties, the moieties are responsible for binding with the target cells. Administering a mixture of multi-valent probes comprise excessive cell-targeting moieties may result in over-crosslinking between the probes and target cells, thereby jeopardizing the imaging resolution. As to multi-valent functional molecules with therapeutic moieties, one cannot ascertain the administered dosage without knowing the exact number of therapeutic moieties comprised therein.

Accordingly, there exists a need in the related art a simplified method for preparing single-valent functional molecules.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

According to various aspects and embodiments of the present disclosure, the present invention provides a facile method for the preparation of single-valent avidin-biotin-peptide conjugates, which can be used as a platform to produce a wide range of functional conjugates.

In one aspect, the present disclosure is directed to a method for preparing single-valent, modified avidin-like molecules.

According to one embodiment of the present disclosure, the method comprises the steps as follows. A plurality of biotinylated peptides are gradually added into a plurality of avidin-like molecules in a molar ratio between 1:4 to 16:1 so as to form a plurality of avidin-biotin-peptide conjugates with different valence states, wherein each of the plurality of biotinylated peptides comprises a His-tag sequence. Next, the plurality of avidin-biotin-peptide conjugates are separated by use of a nickel affinity column eluted with an imidazole gradient to produce a first eluate, wherein the first eluate consists essentially of a plurality of single-valent avidin-biotin-peptide conjugates, in which each conjugate has a first valence.

According to various embodiments of the present disclosure, the number of the plurality of biotinylated peptides that is added into the plurality of avidin-like molecules per minute is 1% to 10% of the total number of the plurality of avidin-like molecules. Preferably, the number of the biotinylated peptides added into the avidin-like molecules per minute is 2% to 6% of the total number of the avidin-like molecules.

According to various embodiments of the present disclosure, the avidin-like molecule is avidin, streptavidin, NeutrAvidin, or CaptAvidin. For example, the avidin-like molecule is avidin or streptavidin.

In some optional embodiments, the biotinylated peptide comprises an amino acid sequence of SEQ ID No. 1, and the C-terminus of the amino acid sequence is biotinylated.

In some other embodiments, the biotinylated peptide comprises an amino acid sequence of SEQ ID No. 2, and the N-terminus of the amino acid sequence is biotinylated.

Still optionally, the biotinylated peptide further comprises an amino acid sequence of a cell-targeting sequence, a cell-penetrating sequence, or a therapeutic sequence, locating at the N-terminus or C-terminus of the His-tag sequence.

According to various embodiments of the present disclosure, the imidazole gradient is 5 mM to 1,500 mM imidazole.

In another aspect, the present disclosure is directed to a method for preparing a plurality of functional conjugates.

According to one embodiment of the present disclosure, the method comprises the steps as follows. A plurality of biotinylated peptides are gradually added into a plurality of avidin-like molecules in a molar ratio between 1:4 to 16:1 so as to form a plurality of avidin-biotin-peptide conjugates with different valence states, wherein each of the plurality of biotinylated peptides comprises a His-tag sequence. Next, the plurality of avidin-biotin-peptide conjugates are separated by use of a nickel affinity column eluted with an imidazole gradient to produce at least one eluate, wherein the at least one eluate comprises a first eluate consisting essentially of a plurality of first single-valent avidin-biotin-peptide conjugates, in which each conjugates has a first valence. The plurality of first single-valent avidin-biotin-peptide conjugates are then mixed with a plurality of first functional molecules under conditions allowing the formation of a plurality of first avidin-biotin-peptide/first functional molecule conjugates.

In certain embodiments of the present disclosure, each first functional molecule comprises a first functional moiety, which, for example, may be selected from the group consisting of, nucleotides, peptides, antibodies, antigens, nanoparticles, quantum dots, fluorescent materials, magnetic materials, and therapeutics.

In certain optional embodiments, each first functional molecule comprises a reaction group capable of reacting with the avidin-like molecules, thereby binding with the avidin-like molecules. An illustrative example of such reaction group is carboxyl group (—COOH).

In certain optional embodiments, the present method further comprises the steps as follows. A plurality of second functional molecules are mixed with the plurality of first avidin-biotin-peptide/first functional molecule conjugates so as to form a plurality of first avidin-biotin-peptide/first functional molecule/second functional molecule conjugates, wherein each of the plurality of the second functional molecules comprises a second avidin-binding moiety or a second His-tag-binding moiety.

In certain embodiments of the present disclosure, each second functional molecule also comprises a second functional moiety covalently bound to the second avidin-binding moiety or second His-tag-binding moiety, and the second functional moiety is selected from the group consisting of, nucleotides, peptides, antibodies, antigens, nanoparticles, quantum dots, fluorescent materials, magnetic materials, and therapeutics. The second biotinylated functional molecule can be the same as or different from the first biotinylated functional molecule.

In alternative embodiments, each first functional molecule comprises an avidin-binding moiety or a His-tag-binding moiety, and the molar ratio between the plurality of first functional molecules and the plurality of single-valent avidin-biotin-peptide conjugates 1:4 to 16:1.

Each of the plurality of first functional molecules comprises a first avidin-binding or a His-tag-binding moiety, so that the first functional molecules bind to either the biotin moiety or His-tag moiety of the first single-valent avidin-biotin-peptide conjugates. According to various embodiments of the present disclosure, the number of the plurality of first functional molecules that is added into the plurality of first single-valent avidin-biotin-peptide conjugates per minute is 1% to 10% of the total number of the plurality of first single-valent avidin-biotin-peptide conjugates. Preferably, the number of the plurality of first functional molecules that is added into the plurality of first single-valent avidin-biotin-peptide conjugates per minute is 2% to 6% of the total number of the plurality of first single-valent avidin-biotin-peptide conjugates.

According to various embodiments of the present disclosure, the number of the plurality of biotinylated peptides that is added into the plurality of avidin-like molecules per minute is 1% to 10% of the total number of the plurality of avidin-like molecules. Preferably, the number of biotinylated peptides added into the avidin-like molecules per minute is 2% to 6% of the total number of the avidin-like molecules.

In certain optional embodiments, the at least one eluate further comprises a second eluate consisting essentially of a plurality of second single-valent avidin-biotin-peptide conjugates, in which each conjugates has a second valence that is different from the first valence; and the present method further comprises the step of mixing the plurality of second single-valent avidin-biotin-peptide conjugates with a plurality of second functional molecules under conditions allowing the formation of a plurality of second avidin-biotin-peptide/second functional molecule conjugates.

The above-mentioned second functional molecule, like the first functional molecule, comprises a second functional moiety selected from the group consisting of, nucleotides, peptides, antibodies, antigens, nanoparticles, quantum dots, fluorescent materials, magnetic materials, and therapeutics. Also, the second functional molecule may comprise any of a reaction group, avidin-binding moiety, and His-tag-binding moiety.

According to various embodiments of the present disclosure, the avidin-like molecule is avidin, streptavidin, NeutrAvidin, or CaptAvidin. For example, the avidin-like molecule is avidin or streptavidin.

In some optional embodiments, the biotinylated peptide comprises an amino acid sequence of SEQ ID No. 1, and the C-terminus of the amino acid sequence is biotinylated.

In some other embodiments, the biotinylated peptide comprises an amino acid sequence of SEQ ID No. 2, and the N-terminus of the amino acid sequence is biotinylated.

Still optionally, the biotinylated peptide further comprises an amino acid sequence of a cell-targeting sequence or a cell-penetrating sequence, locating at the N-terminus or C-terminus of the His-tag sequence.

According to various embodiments of the present disclosure, the imidazole gradient is 5 mM to 1,500 mM imidazole.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1A and FIG. 1B are schematic diagrams respectively illustrating methods for preparing single-valent, modified avidin-like molecules according to certain embodiments of the present disclosure;

FIGS. 2A, 2B, 3A, and 3B are schematic diagrams respectively illustrating methods for preparing modified functional conjugates according to some embodiments of the present disclosure;

FIG. 4A to FIG. 4C are photographs of electrophoresis results according to Example 1 of the present disclosure;

FIG. 5 to FIG. 8 are photographs of electrophoresis results according to Example 2 of the present disclosure;

FIG. 9 is a photograph of electrophoresis results according to Example 3 of the present disclosure;

FIG. 10A to FIG. 10C are photographs of electrophoresis results according to Example 4 of the present disclosure;

FIG. 11 is a photograph of electrophoresis results according to Example 5.2 of the present disclosure; lane C1: 10 μL of 1.5 μM 6 nm-AuNPs and 10 μL of double distilled water; lane C2: 10 μL of 1.5 μM 6 nm-AuNPs and 10 μL of 2 μM streptavidin solution; lane C3: 10 μL of 1.5 μM Biotin-PEG₍₅₀₀₀₎@AuNPs_{(6 nm)} and 10 μL of double distilled water; and lanes 1 to 6: 10 μL of 1.5 μM Biotin-PEG₍₅₀₀₀₎@AuNPs_{(6 nm)} reacted with 10 μL of streptavidin solution at the concentration indicated in parentheses;

FIG. 12 is a photograph of electrophoresis results according to Example 5.2 of the present disclosure; lane C1: 10 μL of 1.5 μM 6 nm-AuNPs and 10 μL of double distilled water; lane C2: 10 μL of 2 μM Biotin-PEG₍₅₀₀₀₎@AuNPs_{(6 nm)} and 10 μL of double distilled water; lane 1: 10 μL of 2 μM Biotin-PEG₍₅₀₀₀₎@AuNPs_{(6 nm)} reacted with 10 μL of 0.5 μM streptavidin solution; lane 2: 10 μL of 2 μM Biotin-PEG₍₅₀₀₀₎@AuNPs_{(6 nm)} reacted with 10 μL of 0.1 μM mono-valent avidin-biotin-peptide conjugates; lane 3: 10 μL of 2 μM Biotin-PEG₍₅₀₀₀₎@AuNPs_{(6 nm)} reacted with 10 μL of 0.2 μM bi-valent avidin-biotin-peptide conjugates; lane 4: 10 μL of 2 μM Biotin-PEG₍₅₀₀₀₎@AuNPs_{(6 nm)} reacted with 10 μL of 0.5 μM tri-valent avidin-biotin-peptide conjugates; and lane 5: 10 μL of 2 μM Biotin-PEG₍₅₀₀₀₎@AuNPs_{(6 nm)} reacted with 10 μL of 0.5 μM tetra-valent avidin-biotin-peptide conjugates;

FIG. 13A to FIG. 13D are Atomic Force Microscopic (AFM) images of functional conjugates prepared in Example 5.2 of the present disclosure;

FIG. 14 is a photograph of electrophoresis results according to Example 5.4 of the present disclosure; lane C1: 1 μM polymer-coated 6 nm-AuNPs; lane C2: 1 μM 6 nm-AuNP/tri-valent avidin-biotin-peptide conjugates; lane C3: 50 nM polymer-coated 15 nm-AuNPs; lane C4: 40 nM biotinylated 15 nm-AuNPs; lane C5: 1 μM 6 nm-AuNP/streptavidin; lanes 1 to 6: 30 nM biotinylated 15 nm-AuNPs conjugated with 0.8, 0.4, 0.2, 0.1, 0.05, and 0.025 μM polymer-coated 6 nm-AuNPs, respectively; and lanes 7 to 12: 30 nM biotinylated 15 nm-AuNPs conjugated with 0.8, 0.4, 0.2, 0.1, 0.05, and 0.025 μM 6 nm-AuNP/streptavidin; and

FIG. 15 is a transmission electron microscopic (TEM) image of modified functional conjugates prepared in Example 5.4 of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless defined otherwise, all technical and scientific terms used herein have the same meaning as commonly understood by one of the ordinary skill in the art to which this invention belongs.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

As used herein, the term “biotinylated” or “biotinylation” refers to the addition to a molecule of one or more molecules of biotin, a biotin analog or derivative (e.g., diaminobiotin, 2-iminobiotin, biocytin, desthiobiotin, bisnorbiotin, tetranorbiotin, biotin sulfoxide, biotin sulfone, iminobiotin trifluoroacetamide, biotin-triethylene glycol, or a cleavable biotin analog), or any other reagent that is capable of binding to an avidin-like molecule. Biotinylation of a particular molecule can be achieved using methods that are well-known in the art.

Here, the term “avidin-binding moiety” refers to a moiety capable of binding to an avidin-like molecule. For example, the avidin-binding moiety may bind with a molecule via the above-mentioned biotinylation.

The term “His-tag-binding moiety” as used herein refers to a moiety capable of binding to a His-tag sequence. For example, nitrilotriacetic acid (NTA), NTA analogs, NTA derivatives, and NTA-containing molecules are known His-tag-binding moieties.

The term “cell-targeting sequence” as used herein refers to any amino acid sequence that targets a particular cell type, such as a cancer cell. Such cell-targeting sequences can target a cell by interacting with, or binding to, cell-surface receptors or other molecules on the cell surface. Alternatively, the cell-targeting sequence can also target a cell by interacting with proteins secreted by the particular cell.

As used herein, the term “cell-penetrating sequence” refers to an amino acid sequence that can readily cross a biological membrane (such as, the phospholipid bilayer, mitochondrial, endosomal and nuclear membrane) or a physiological barrier (e.g., the blood-brain, trans-mucosal, hematoretinal, skin, gastrointestinal and pulmonary barriers).

Here, the “therapeutic sequence” refers to an amino acid sequence that is capable of exhibiting a therapeutic effect in a subject.

According to principles and spirits of the present disclosure, avidin-biotin-peptide conjugates of different valences are formed by controlling the ratio of the avidin-like molecules and biotinylated peptides in a reaction system. Thereafter, avidin-biotin-peptide conjugates of the same valence are separated from the reaction system by use of a nickel affinity column. The thus-obtained single-valent avidin-biotin-peptide conjugates may serve as a platform with both biotin and NTA functionalities that may react with functional molecules having either a biotin-binding or His-tag-binding moiety to form modified functional conjugates. Alternatively, the present single-valent avidin-biotin-peptide conjugates may have one or more reaction groups capable of reacting with one or more reaction groups of functional molecules, thereby producing modified functional conjugates.

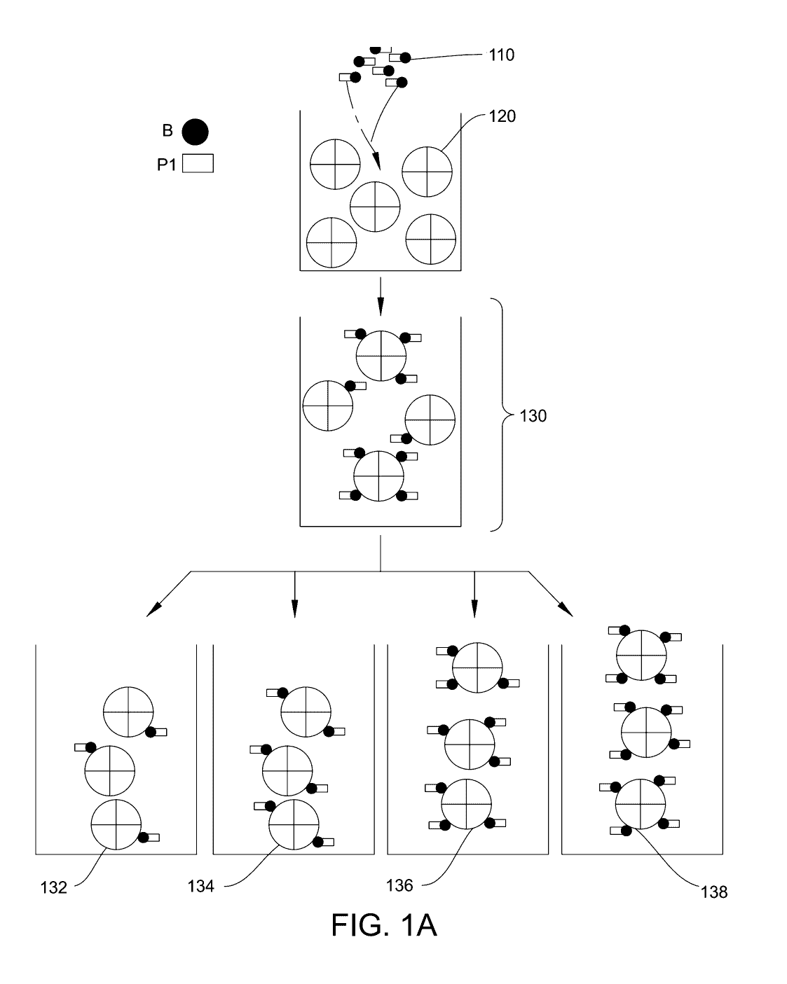

The method for preparing single-valent, modified avidin-like molecules according to one aspect of the present disclosure is discussed with reference to FIG. 1A.

First, a plurality of first biotinylated peptides 110 are gradually added into a plurality of avidin-like molecules 120 to form a first mixture 130 of a plurality of avidin-biotin-first peptide conjugates having different valence states.

As is evident from the working examples provided below, it is important to control the ratio between the first biotinylated peptides 110 and the avidin-like molecules 120 in the reaction system so that the avidin-biotin-first peptide conjugates having various valence states are synthesized. According to various embodiments of the present disclosure, this could be achieved by controlling the total molar ratio of the two reactants and/or the rate at which the two reactants are mixed.

Therefore, in certain embodiments of the present disclosure, the molar ratio between the first biotinylated peptides 110 and the avidin-like molecules 120 is 1:4 to 16:1. For example, said molar ratio can be 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, or a value in the range between any two of the above-mentioned values.

In some optional embodiments, the first biotinylated peptides 110 are added into the avidin-like molecules 120 at a rate where, for each minute, the number of the first biotinylated peptides 110 added into the reaction system is 1% to 10% of the total number of the avidin-like molecules 120. In other words, for every one hundred avidin-like molecules 120 in the reaction system, 1 to 10 first biotinylated peptides 110 are added therein per minute. In certain embodiments, the number of the first biotinylated peptides 110 added into the reaction system per minute is 2% to 6% of the total number of the avidin-like molecules 120. For example, the number of the first biotinylated peptides 110 added into the reaction system per minute is 1, 2, 3, 4, 5, 6, 7, 8, 9, or 10% (or a value in the range between any two of the above-mentioned values) of the total number of the avidin-like molecules 120.

As could be appreciated, each of the first biotinylated peptides 110 comprises a first amino acid sequence (P1) and a biotin molecule (B) that is covalently attached to the N-terminus or C-terminus of the first amino acid sequence (P1).

According to various embodiments of the present disclosure, each of the first biotinylated peptides 110 comprises a His-tag sequence to facilitate the subsequent separation procedure. For example, the His-tag sequence may consist of 4 to 12 histidine residues; however, the present disclosure is not limited thereto.

In some optional embodiments, the first biotinylated peptide 110 comprises an amino acid sequence of SEQ ID No. 1, and the C-terminus of the amino acid sequence is biotinylated. In other optional embodiments, the first biotinylated peptide 110 comprises an amino acid sequence of SEQ ID No. 2, and the N-terminus of the amino acid sequence is biotinylated.

According to certain optional embodiments, the first biotinylated peptide 110 further comprises an amino acid sequence of a cell-targeting sequence, a cell-penetrating sequence, or a therapeutic sequence, locating at the N-terminus or C-terminus of the His-tag sequence.

According to various embodiments of the present disclosure, the avidin-like molecule 120 has a tetrameric structure that possesses one biotin-binding site per monomeric unit. Illustrative examples of the avidin-like molecule 120 include avidin, streptavidin, NeutrAvidin, and CaptAvidin. According to certain working examples of the present disclosure, the avidin-like molecule 120 is avidin or streptavidin.

After the formation of the first mixture 130, the plurality of avidin-biotin-first peptide conjugates within the first mixture 130 are separated based on their respective valences to produce a first eluate consisting essentially of a plurality of single-valent avidin-biotin-first peptide conjugates, in which each conjugates has a first valence.

Specifically, the separation procedure is carried out by use of a first nickel affinity column eluted with a first imidazole gradient. According to various embodiments of the present disclosure, the first imidazole gradient may comprise two or more imidazole solutions of different concentrations in the range of 5 mM to 1,500 mM; preferably in the range of 10 mM to 1,000 mM; and more preferably in the range of 25 mM to 500 mM. For example, the concentration of the imidazole solution used in the first imidazole gradient may be 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1,000, 1,050, 1,100, 1,150, 1,200, 1,250, 1,300, 1,350, 1,400, 1,450, 1,500 mM, or a value in the range between any two of the above-mentioned concentrations.

After eluting with the first imidazole gradient, a first eluate consisting essentially of a plurality of single-valent avidin-biotin-first peptide conjugates respectively having a first valence is produced. For example, as illustrated in FIG. 1A, the single-valent avidin-biotin-first peptide conjugates may be mono-valent avidin-biotin-first peptide conjugates 132, bi-valent avidin-biotin-first peptide conjugates 134, tri-valent avidin-biotin-first peptide conjugates 136, or tetra-valent avidin-biotin-first peptide conjugates 138.

In certain optional embodiments, the above-mentioned single-valent avidin-biotin-first peptide conjugates may be further modified with one or more second biotinylated peptides. In this case, the single-valent avidin-biotin-first peptide conjugates should have at least one vacant biotin-binding site. For example, the single-valent avidin-biotin-first peptide conjugates may be the above-mentioned mono-valent avidin-biotin-first peptide conjugates 132, bi-valent avidin-biotin-first peptide conjugates 134, or tri-valent avidin-biotin-first peptide conjugates 136.

Referring to FIG. 1B, the method for modifying the single-valent avidin-biotin-first peptide conjugates is described using the mono-valent avidin-biotin-first peptide conjugates 132 as an example. However, as could be appreciated, the present invention is not limited thereto; rather, the either the bi-valent avidin-biotin-first peptide conjugates 134 or tri-valent avidin-biotin-first peptide conjugates 136 can be used in following embodiments.

First, a plurality of second biotinylated peptides 140 are gradually added into the plurality of single-valent avidin-biotin-first peptide conjugates (in this case, the mono-valent avidin-biotin-first peptide conjugates 132) to form a 10o second mixture 150 of a plurality of avidin-biotin-first peptide/second peptide conjugates with different valence states.

According to various embodiments of the present disclosure, the molar ratio between the mono-valent avidin-biotin-first peptide conjugates 132 and the second biotinylated peptides 140 is 1:4 to 16:1. For example, said molar ratio can be 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, or a value in the range between any two of the above-mentioned values.

In some optional embodiments, the second biotinylated peptides 140 are added into the mono-valent avidin-biotin-first peptide conjugates 132 at a rate where, for each minute, the number of the first biotinylated peptides 110 added into the reaction system is 1% to 10% of the total number of the mono-valent avidin-biotin-first peptide conjugates 132. In other words, for every one hundred mono-valent avidin-biotin-first peptide conjugates 132 in the reaction system, 1 to 10 second biotinylated peptides 140 are added therein per minute. In certain embodiments, the number of the second biotinylated peptides 140 added into the reaction system per minute is 2% to 6% of the total number of the mono-valent avidin-biotin-first peptide conjugates 132. For example, the number of the second biotinylated peptides 140 added into the reaction system per minute is 1, 2, 3, 4, 5, 6, 7, 8, 9, or 10% (or a value in the range between any two of the above-mentioned values) of the total number of the mono-valent avidin-biotin-first peptide conjugates 132.

Like the first biotinylated peptide 110 described hereinabove, the second biotinylated peptide 140 comprises a second amino acid sequence (P2) and a biotin molecule (B) that is covalently attached to the N-terminus or C-terminus of the second amino acid sequence (P2).

Therefore, the second biotinylated peptide 140 may also comprise a His-tag sequence, as described above regarding the first biotinylated peptide 110. Similarly, the above description concerning the sequence of the first biotinylated peptide 110 is also applicable in the present embodiments regarding 15s the second biotinylated peptide 140. According to embodiments of the present disclosure, the sequence of the second amino acid sequence (P2) is different from that of the first amino acid sequence (P1).

After the formation of the second mixture 150, the plurality of avidin-biotin-first peptide/second peptide conjugates within the second mixture 150 are separated based on their respective valence to produce a second eluate consisting essentially of a plurality of single-valent avidin-biotin-first peptide/second peptide conjugates with a second valence.

Specifically, the separation procedure is carried out with by use of a second nickel affinity column eluted with a second imidazole gradient. According to various embodiments of the present disclosure, the second imidazole gradient may comprise two or more imidazole solutions of different concentration in the range of 5 mM to 1,500 mM; preferably in the range of 10 mM to 1,000 mM; and more preferably in the range of 25 mM to 500 mM. As could be appreciated, the above-listed imidazole solutions for use in the first imidazole gradient are also suitable to be used in the present second imidazole gradient.

After eluting with the second imidazole gradient, a second eluate consisting essentially of a plurality of single-valent avidin-biotin-first peptide/second peptide conjugates respectively having second valences, is produced. For example, as illustrated in FIG. 1B, the single-valent avidin-biotin-first peptide/second peptide conjugates may be 1P1/1P2 conjugates 152 in which one monomeric unit thereof is conjugated with the first peptide (P1), another monomeric unit thereof is conjugated with the second peptide (P2), while the remaining two monomeric units are vacant. As could be appreciated the single-valent avidin-biotin-first peptide/second peptide conjugates may also be 1P1/2P2 conjugates 154 or 1P1/3P2 conjugates 156 illustrated in FIG. 1B.

According to other embodiments of the present disclosure, when the bi-valent avidin-biotin-first peptide conjugates 134 are used as the starting materials, the resulting single-valent avidin-biotin-first peptide/second peptide conjugates may be 2P1/1P2 conjugates or 2P1/2P2 conjugates. On the other hand, when the tri-valent avidin-biotin-first peptide conjugates 136 are used as the starting materials in the present method, the resulting single-valent avidin-biotin-first peptide/second peptide conjugates may be 3P1/1 P2 conjugates.

As discussed above, the conjugates obtained by the above-mentioned methods could be used as a platform for preparing modified functional conjugates. Accordingly, methods for preparing modified functional conjugates using the above-mentioned single-valent, modified avidin-like molecules fall within the scope of another aspect of the present disclosure.

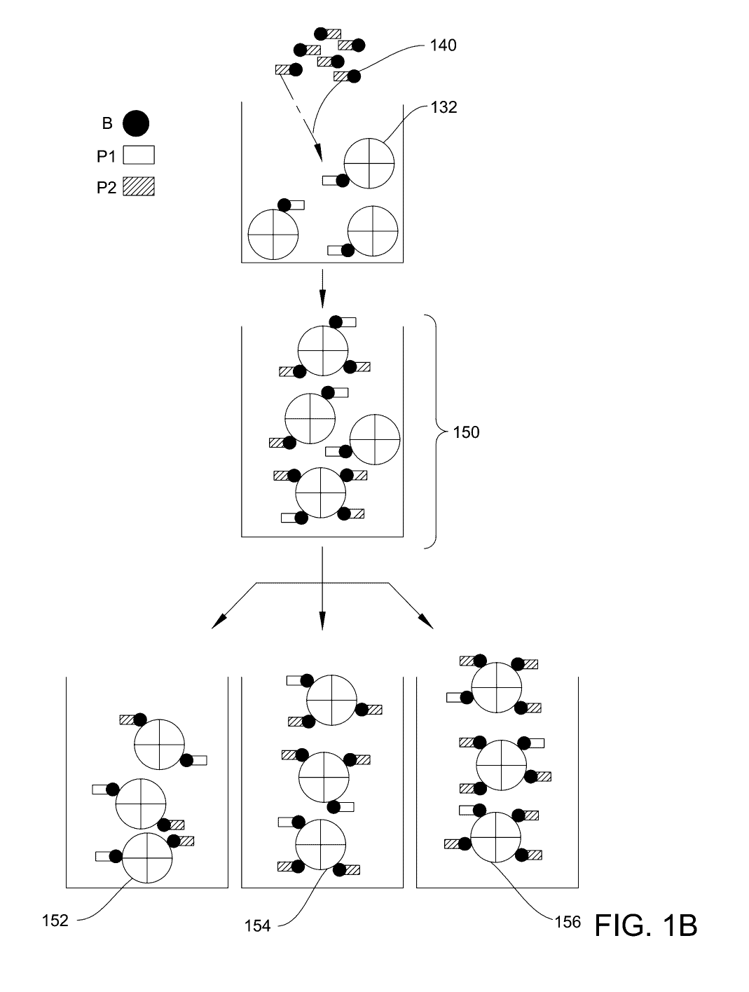

According to certain embodiments of the present disclosure, methods for preparing modified functional conjugates through biotin-binding are discussed below in connection with FIG. 2A.

According to various embodiments of the present disclosure, a plurality of single-valent, modified avidin-like molecules are first prepared. Specifically, the single-valent, modified avidin-like molecules are prepared by methods according to embodiments of the first aspect of the present disclosure. For the purpose of discussion, the mono-valent avidin-biotin-first peptide conjugates 132 are illustrated in FIG. 2A as the single-valent, modified avidin-like molecules; however, other single-valent, modified avidin-like molecules having at least one vacant biotin-binding site can also be used in the present method. Examples of the such single-valent, modified avidin-like molecules include, but are not limited to, the mono-valent avidin-biotin-first peptide conjugates 132, bi-valent avidin-biotin-first peptide conjugates 134, or tri-valent avidin-biotin-first peptide conjugates 136 of FIG. 1A; the 1P1/1P2 conjugates 152 or 1P1/2P2 conjugates 154 of FIG. 1B; or the 2P1/1P2 conjugates (not illustrated).

Thereafter, the single-valent, modified avidin-like molecules (in this case, the mono-valent avidin-biotin-first peptide conjugates 132) are mixed with a plurality of first functional molecules 210 under conditions allowing the formation of a plurality of first modified functional conjugates 220. As illustrated, the first functional molecule 210 comprises a first functional moiety (F1) and a biotin-binding moiety (B). In this case, each biotin-binding moiety (B) may bind with the one of the vacant biotin-binding sites (V) of the mono-valent avidin-biotin-first peptide conjugates 132, thereby forming the first modified functional conjugates 220.

In certain embodiments of the present disclosure, the first functional moiety (F1) is selected from the group consisting of, nucleotides, peptides, antibodies, antigens, nanoparticles, quantum dots, fluorescent materials, magnetic materials, and therapeutics.

As illustrated in FIG. 1A, the elution with the first imidazole gradient may produce more than one eluates. Accordingly, in certain optional embodiments, the second eluate consisting essentially of a plurality of second single-valent avidin-biotin-peptide conjugates respectively having second valences (for example, the bi-valent avidin-biotin-peptide conjugates 134 illustrated in FIG. 2B) are mixed with a plurality of second biotinylated functional molecules 230 under conditions allowing the formation of a plurality of second modified functional conjugates 240. As illustrated in FIG. 2B, each bi-valent avidin-biotin-peptide conjugate 134 has two vacant biotin-binding sites (V); after reaction, two second biotinylated functional molecules 230 are respectively attached to said two vacant biotin-binding sites (V), and hence each 20 second modified functional conjugate 240 has two first peptide moieties (P1) and two second functional moieties (F2). However, the present disclosure is not limited thereto, and as discussed above, modified functional conjugates having two first peptide moieties (P1) and one second functional moiety (F2) may be prepared by controlling the reaction conditions used.

According to various embodiments of the present disclosure, the second functional moiety (F2) may be the same as or different from the first functional moiety (F1). Similarly, the second functional moiety (F2) may be selected from the group consisting of, nucleotides, peptides, antibodies, antigens, nanoparticles, quantum dots, fluorescent materials, magnetic materials, and therapeutics.

In addition to conjugation through the binding between the avidin-like moiety of the single-valent avidin-biotin-first peptide conjugate and the biotin moiety of the functional molecule, the single-valent avidin-biotin-first peptide conjugates may also react with functional molecules with a His-tag-binding moiety to form modified functional conjugates, according to embodiments of the present disclosure.

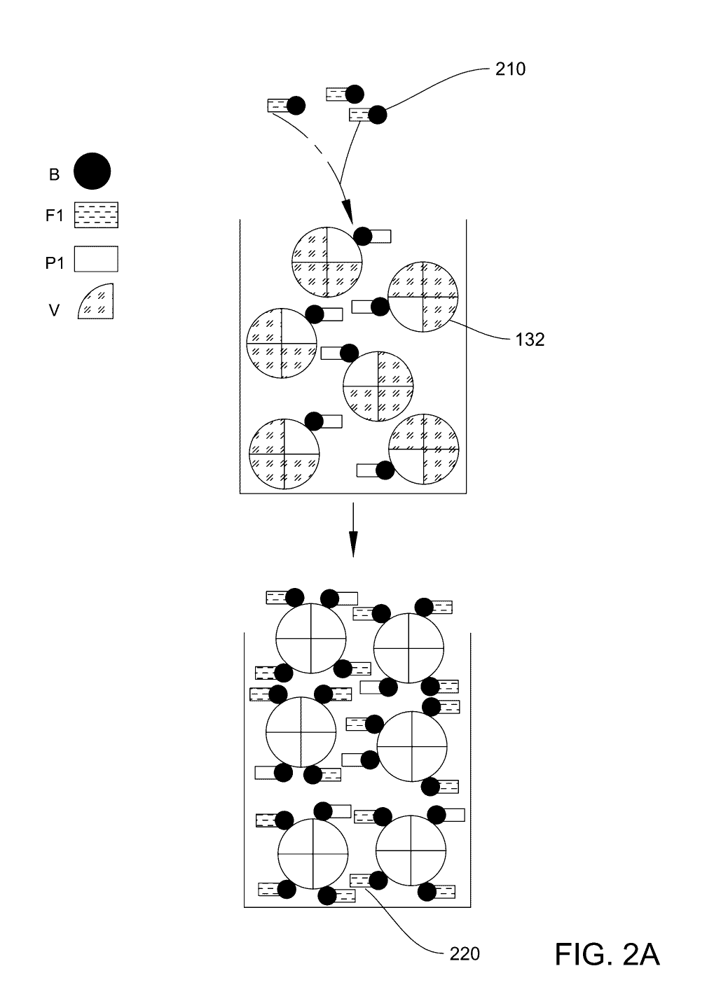

In FIG. 3A, each of the third functional molecules 310 comprises an NTA moiety (N) and a third functional moiety (F3). The third functional molecules 310 are mixed with the mono-valent avidin-biotin-first peptide conjugates 132 under conditions that allow the formation of a plurality of third modified functional conjugates 320.

Since the third functional molecule 310 comprises the NTA moiety (N) capable of binding with the His-tag of the mono-valent avidin-biotin-first peptide conjugates 132, the vacant biotin-binding sites (V) of the mono-valent avidin-biotin-first peptide conjugate 132 remain vacant in the third modified functional conjugate 320. Accordingly, such third modified functional conjugates 320 may be subjected to the above-mentioned methods described in connection with FIG. 2A and/or FIG. 2B. Likewise, the products produced from the methods of FIG. 2A and/or FIG. 2B are also suitable to be used in the present method of FIG. 3A.

Grafting of a functional molecule onto the present single-valent avidin-biotin-peptide conjugates may also be achieved through the bonding between the reaction group of the functional molecule and the reaction group of the single-valent avidin-biotin-peptide conjugates. For example, it is known that the avidin-like molecule has an amine group (−NH₂), and when the functional molecule possess a reaction group capable of reacting with the amine group, the modification of the functional molecule can realized through the reaction between the amine group and the reaction group. FIG. 3B provides an illustrative example for preparing modified functional conjugates through this scheme.

As illustrated in FIG. 3B, the fourth functional molecules 330 are mixed with bi-valent avidin-biotin-first peptide conjugates 134. In this case, the fourth functional molecules 330 do not comprises either a biotin-binding moiety or a His-tag-binding moiety. Rather, the functional moiety F4 of each fourth functional molecule 330 has a reaction group (such as a carboxyl group) capable of reacting with the amine group of the avidin-like molecule. According to certain embodiments of the present disclosure, the fourth functional molecule 330 may be nanoparticles (e.g., gold nanoparticles) with amphiphilic polymer. After reaction under suitable conditions, a plurality of fourth functional conjugates 332 are produced. As could be appreciated, since the fourth functional molecule 330 des not bind with the avidin-like molecule via the avidin-biotin binding, each fourth functional conjugates 332 still has two vacant biotin-binding sites (V).

Optionally, the fourth functional conjugates 332 may be subjected to further modification. For example, in FIG. 3B, the fourth functional conjugates 332 are mixed with a plurality of second biotinylated functional molecules 230. In this scenario, since each fourth functional conjugates 332 has two vacant biotin-binding sites (V), up to two second biotinylated functional molecules 230 may be conjugated with one fourth functional conjugates 332. In this way, a plurality of fifth functional conjugates 350 are produced, and each of the fifth functional conjugates 350 has two attached second biotinylated functional molecules 230. However, as could be appreciated, modified functional conjugates having two first peptide moieties (P1) and one second functional moiety (F2) may also be prepared by controlling the reaction conditions used.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example 1

### Preparation of Avidin-Biotin-Peptide Conjugates

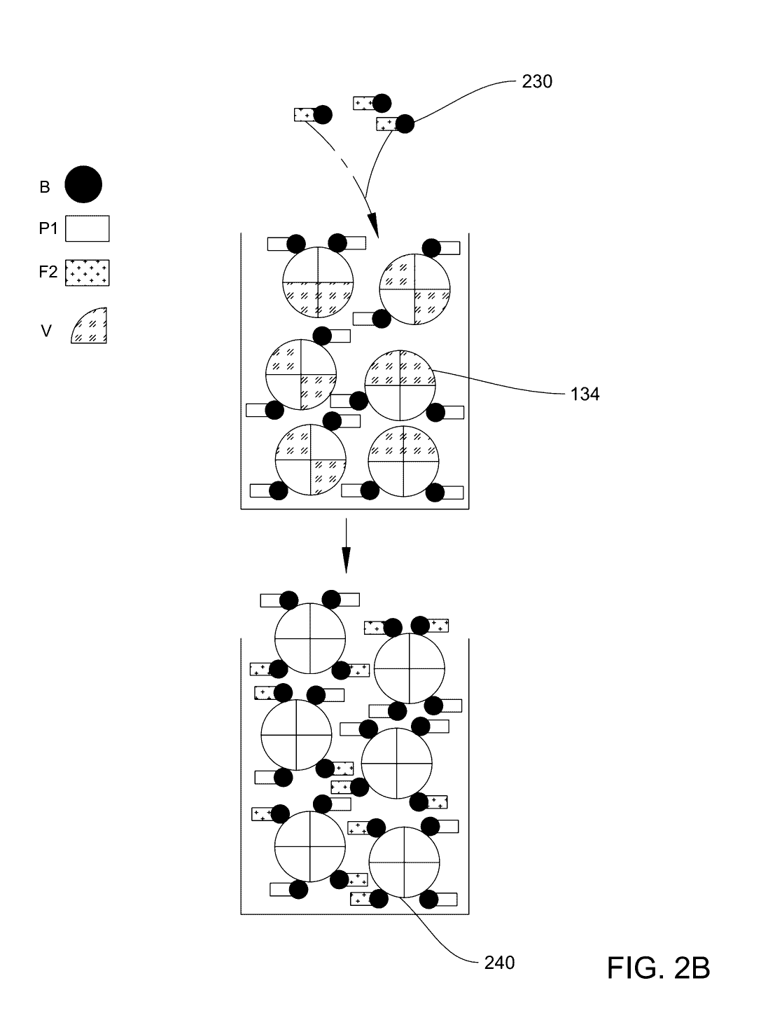

Biotinylated peptide conjugates including His×6-G×4-Biotin (95%) (SEQ IUD No. 1) and Biotin-G-G-His×6 (95%) (SEQ ID No. 2) were purchased from MDBio Inc. (Taipei, Taiwan, ROC). Biotinylated peptide conjugates were serially diluted in double distilled water (5 μM to 320 μM, 2-fold serial dilution) to produce biotinylated peptide solutions of various concentrations. D-Biotin was also serially diluted in double distilled water to produce 5 μM to 320 μM biotin solutions. 20 μM streptavidin solution was prepared by dissolving 7.9 μg salt-free streptavidin (Sigma 85878) in 7.5 μL of double distilled water. Then, 7.5 μL of streptavidin solution was mixed with 7.5 μL of biotinylated peptide solutions or biotin solutions of various concentrations to produce avidin-biotin-peptide conjugates. The products were then subjected to vertical electrophoresis (8% polyacrylamide gel; 150 V; 150 minutes; sample buffer: 5.55 mL of double distilled water, 1.25 mL of Tris-HCL (0.5 M, pH 6.8, Sigma), 3.0 mL of glycerol (UNION Chemical Works Ltd.), and 0.2 mL of 0.5% (v/v) bromophenol blue (Sigma); running buffer: 1.53 g Tris base and 72.5 g glycerol in double distilled water (total volume: 1000 mL), pH 8.3). FIG. 4A to FIG. 4C are photographs of the electrophoresis results. In these drawings (as well as other drawings, unless specified otherwise), the lane SA contained standard streptavidin solution (2 μM; 15 μL). Also, in FIG. 4A to FIG. 4C, each of lanes 1 to 7 was loaded with 7.5 μL of 2 μM streptavidin solution plus 7.5 μL of biotin solutions or biotinylated peptide solutions of the concentration specified in parenthesis underneath each lane.

The photograph in FIG. 4A indicates that the band moved downward as the concentration of D-biotin increased; however, the molecular weight of the D-biotin was too small, and thus the bands could not be separated properly.

In FIG. 4B, the band moved upward as the concentration of His×6-G×4-biotin conjugate increased. As could be noted, since the His×6-G×4 peptide was electrically neutral, the bands were separated solely based on the molecular weight of the molecules. Accordingly, when the concentration of His×6-G×4-biotin conjugate increased, the numbers of His×6-G×4-biotin conjugated per streptavidin molecule increased, thereby resulting in an increased molecular weight, as evidenced by the band moving upward.

The biotin-G-G-His×6 peptide, on the other hand, was negatively charged. Therefore, during the vertical electrophoresis, the bands were separated based on both the molecular weight and the electrical negativity of the molecules. As could be appreciated, when the concentration of biotin-G-G-His×6 conjugate increased, the numbers of biotin-G-G-His×6 conjugated per streptavidin molecule increased, which resulted in the increase of both the molecular weight and electrical negativity of the product. Therefore, as illustrated in FIG. 4C, the band moved downward as the concentration of biotin-G-G-His×6 conjugate increased. Further, a higher resolution was achieved, as compared with the electrophoresis results in FIG. 4B. Accordingly, the negatively charged biotin-G-G-His×6 conjugates were used in the following working examples.

### Example 2

### Preparation and Purification of Single-Valent Avidin-Biotin-Peptide Conjugates

In the present example, two preparation methods (one-step preparation and drop-wise preparation) and two purification tools (vertical electrophoresis and affinity column) were used to develop a suitable process for preparing single-valent avidin-biotin-peptide conjugates.

2.1. One-Step Preparation

In a 40 mL reaction bottle, 800 μL of 20 μM Streptavidin solution was stirred using a stirrer/hotplate (Corning PC-320 Laboratory Hot Plate and Magnetic Stirrer; Corning, Mich., USA) at the maximal speed, and 10 μM biotin-G-G-His×6 solution (total volume: 3, 6, or 10 mL) was poured into the reaction bottle. The mixture was allowed for reaction for 5 minutes.

2.2. Drop-Wise Preparation

In a 40 mL reaction bottle, 800 μL of 20 μM Streptavidin solution was stirred at the maximal speed, and 10 μM biotin-G-G-His×6 solution (total volume: 3, 6, or 10 mL) was added into the reaction bottle at a flow rate of 83 μL per minute. After the exhaustion of the biotin-G-G-His×6 solution, the mixture was allowed for reaction for 5 minutes.

2.3. Electrophoresis Separation

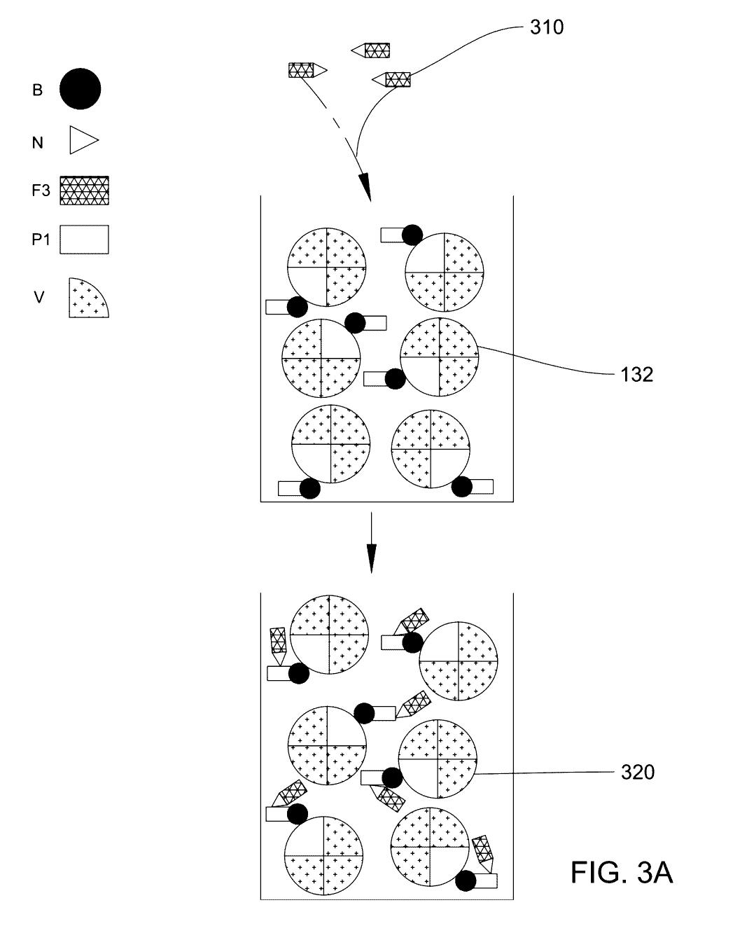

Reaction products produced in Examples 2.1 and 2.2, above, were separated by a first vertical electrophoresis using the parameters set forth in Example 1, above; results are depicted in FIG. 5.

The photograph in FIG. 5 indicates that the one-step preparation process (lanes 1 to 3) could not effectively produced single-valent avidin-biotin-peptide conjugates, especially when the molar ratio between the biotinylated peptides and streptavidin was less than 2:1. Specifically when 3 mL of 10 μM biotinylated peptides were reacted with 0.8 mL of 20 μM streptavidin (molar ratio=1.875:1; see, lane 1, FIG. 5), most of the streptavidin molecules were not conjugated with the biotinylated peptides (band 1), and only a small fraction of streptavidin molecules was conjugated with one (band 2), two (band 3), or three (band 4) biotinylated peptides. On the other hand, when the volume of the biotinylated peptides was increased to 6 mL (see, lane 2, FIG. 5), most of the streptavidin molecules were conjugated with three (band 4) or four (band 5) biotinylated peptides. Similar results were observed in the lane 3 of FIG. 5, in which the starting volume of the biotinylated peptides was 10 mL.

In contrast, lanes 4 to 6 in FIG. 5 establish that the present step-wise preparation process was suitable for preparing single-valent avidin-biotin-peptide conjugates. In particular, when 3 mL of 10 μM biotinylated peptides were gradually mixed with 0.8 mL of 20 μM streptavidin (see, lane 4, FIG. 5), most of the streptavidin molecules was not conjugated (band 1) or conjugated with one biotinylated peptide (band 2). With the increase of the starting volume of the biotinylated peptides, the numbers of non-conjugated streptavidin molecules decreased, and the number of biotinylated peptides that is conjugated with each streptavidin molecule increased. For example, in lane 5 of FIG. 5, most of the streptavidin molecules were conjugated with one (band 2), two (band 3), or three (band 4) biotinylated peptides; while in lane 6 of FIG. 5, most of the streptavidin molecules were conjugated with four biotinylated peptides (band 5).

In view of the foregoing, it is feasible to adjust the valence of the resultant avidin-biotin-peptide conjugates by modifying the relative amounts of the starting materials and/or the rate at which the starting materials are mixed.

2.4. Purification by Vertical Electrophoresis

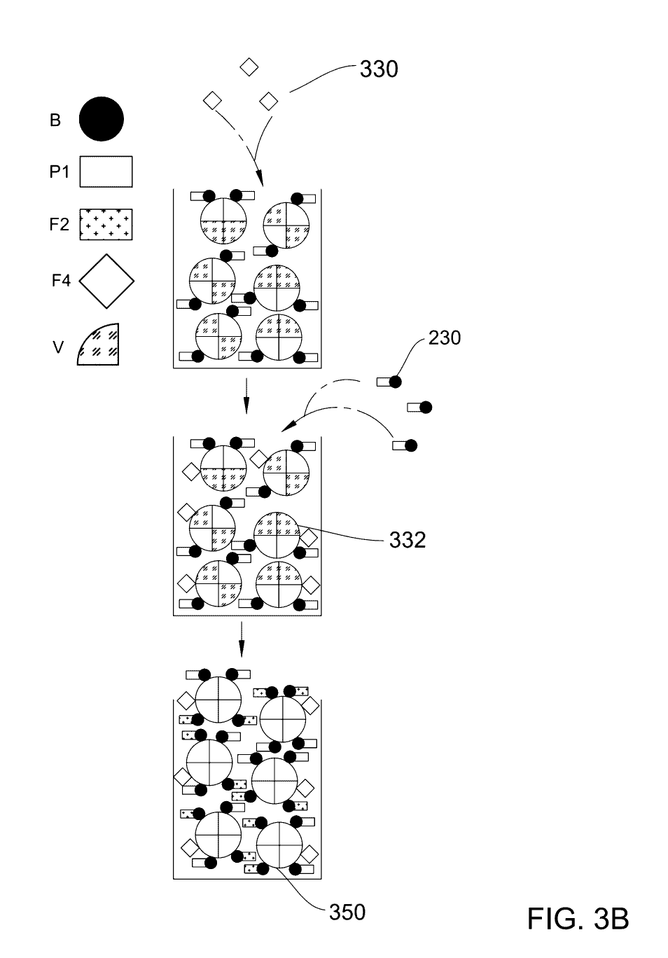

Avidin-biotin-peptide conjugates were prepared using the optimal manufacturing parameters obtained from the above examples (7.5 μL of 20 μM streptavidin and 7.5 μL of 80 μM biotin-G-G-His×6), and then separated using electrophoresis (8% polyacrylamide gel; 150 V; 150 minutes). After separation, the gel strips were excised and eluted by electrophoresis into the dialysis bag (8% polyacrylamide gel; 150 V; 300 minutes). Thereafter, the eluate was concentrated using a 30 kDa molecular sieve. The purification efficiency was analyzed by a vertical electrophoresis (8% polyacrylamide gel; 150 V; 150 minutes); results are depicted in FIG. 6.

In FIG. 6, lanes SA and S were respectively loaded with standard, non-treated streptavidin solution (2 μM; 15 μL) and mixtures of standard single-valent avidin-biotin-peptide conjugates with different valence states (2 μM each; 15 μL), while lanes 1 to 4 contained, respectively, mono-, bi-, tri-, and tetra-valent avidin-biotin-peptide conjugates. The results in FIG. 6 indicate that the vertical electrophoresis could not effectively purify the single-valent avidin-biotin-peptide conjugates depending on the valences thereof. Also, this purification process resulted in substantial loss of the avidin-biotin-peptide conjugates.

2.5. Purification by Affinity Column

The nickel-binding buffer was prepared by mixing 6.065 g Tris base and 17.52 g NaCl in double distilled water (900 mL), adjusted to pH 7.8 with 1 M HCl (total volum: 1,000 mL), and stored at 4° C. until further use.

Imidazole stock solution (500 mM imidazole solution) was prepared by adding 6.065 g Tris base, 17.52 g NaCl and 17.02 g imidazole in double distilled water (900 mL), 1 M HCl was added to adjust the pH value to 7.8; the final volume was 1,000 mL; the stock solution was stored at 4° C. until further use. The stock solution was serially diluted to produce the wash buffer (10 mM imidazole solution) and elution buffers in various concentrations (25, 100, 200, 260, and 500 mM imidazole solution).

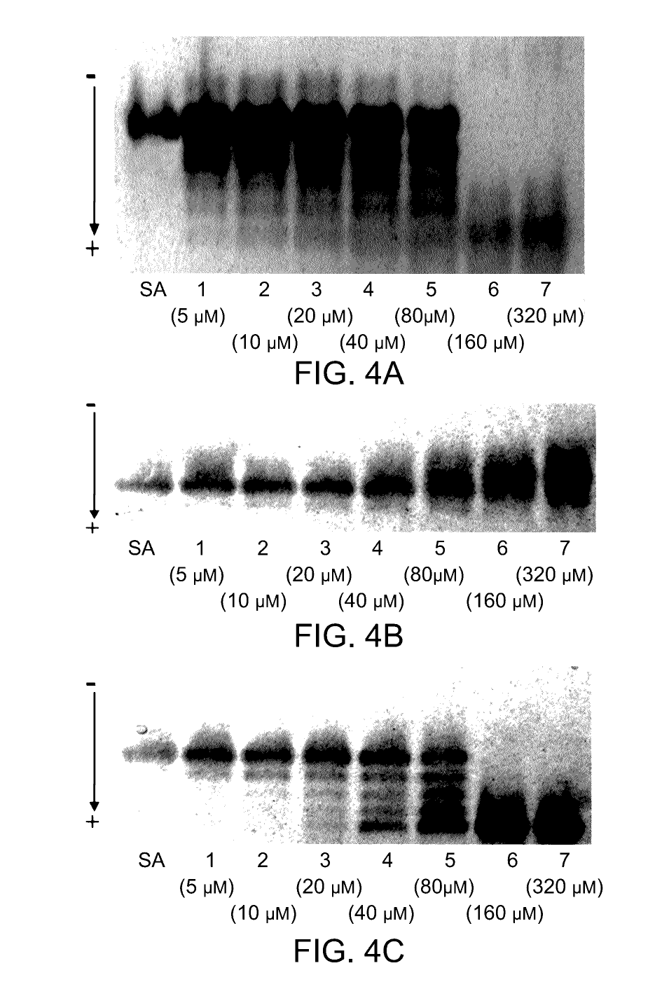

Reaction products (from Example 2.2, using 6 mL of 10 μM biotin-G-G-His×6 solution) were passed through the 30 kDa molecular sieve to remove excessive peptides. Next, 50 μL of samples were loaded into the histidine-tagged (His-tag) affinity column (HisTrap HP, 1 mL, GE) uninterruptedly followed by the addition of ice-cold nickel binding buffer (10 mL) using auto-injector (2 mL per minute). The column was then eluted, in series, with 8 mL of the wash buffer (10 mM imidazole solution), 25 mM imidazole elution buffer, 100 mM imidazole elution buffer, 200 mM imidazole elution buffer, 260 mM imidazole elution buffer, and 500 mM imidazole elution buffer. After each elution, the fractions were collected (5 mL per tube), concentrated with 30 kDa molecular sieve, and confirmed using vertical electrophoresis (8% polyacrylamide gel; 150 V; 150 minutes); results were depicted in FIG. 7.

The results in FIG. 7 demonstrated that the nickel affinity column was more effective in purifying single-valent avidin-biotin-peptide conjugates, compared with vertical electrophoresis (see, FIG. 6). Standard, non-treated streptavidin solution (2 μM; 15 μL; lane SA in FIG. 7) and mixtures of standard single-valent avidin-biotin-peptide conjugates with different valence states (2 μM each; 15 μL; lane S in FIG. 7) were used as controls. Lanes 1 to 4 contained eluates respectively eluted with 100, 200, 260, and 500 mM imidazole elution buffer, while bands 1 to 5 represented non-conjugated streptavidin, and mono-, bi-, tri-, or tetra-valent avidin-biotin-peptide conjugates, respectively.

The results presented in FIG. 7 demonstrated that the non-conjugated streptavidin exhibited the slowest mobility (lane SA, band 1), and the electrophoretic mobility of the conjugate increased as the number of negatively charged peptide conjugated onto the streptavidin increased (lanes 1 to 4) with the tetra-valent avidin-biotin-peptide conjugates being the fastest (lane 4, band 5).

2.6. Binding Affinity Verification

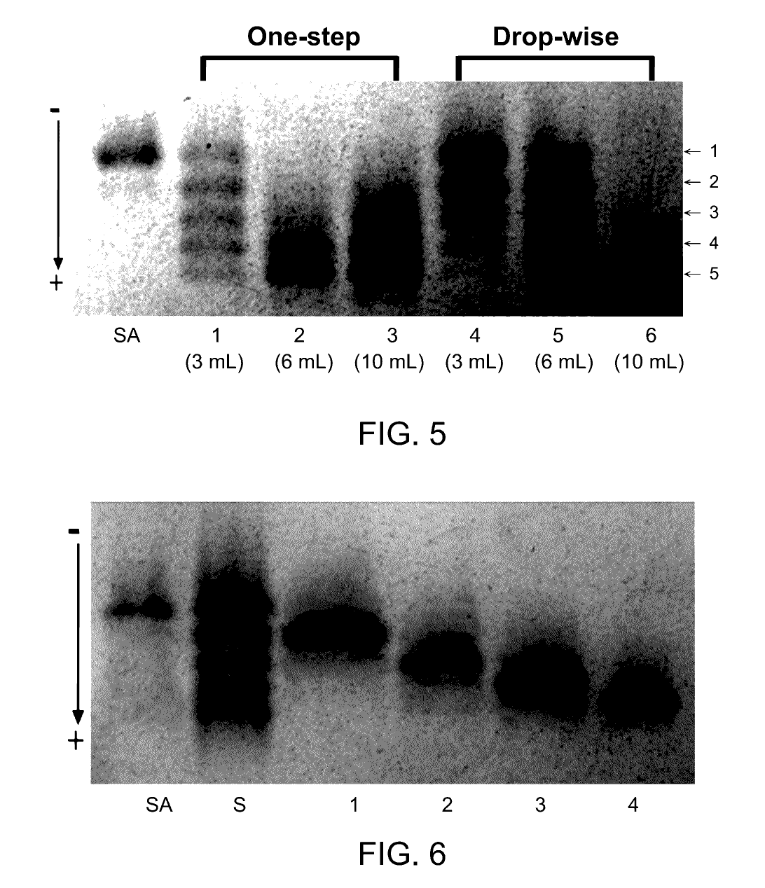

The binding efficacy of the single-valent avidin-biotin-peptide conjugates prepared in Example 2.5, above, was confirmed by reacting the concentrated eluate (2 μM) with excessive amount of 10 mM biotin-G-G-His×6 peptides for 5 minutes, and the products were subjected to vertical electrophoresis (8% polyacrylamide gel; 150 V; 400 mA; 180 minutes); results were depicted in FIG. 8.

In FIG. 8, lanes 1, 3, 5, and 7 respectively contained mono-, bi-, and tri-, or tetra-valent avidin-biotin-peptide conjugates prepared by the step-wise process and purified using nickel affinity column, and lanes 2, 4, 6, or 8 contained the reaction products of mono-, bi-, tri-, or tetra-valent avidin-biotin-peptide conjugates with excessive biotin-G-G-His×6 peptides. As could be seen in FIG. 8, samples containing mono-valent avidin-biotin-peptide conjugates banded at the position of band 2 (lane 1), and after reacting with biotin-G-G-His×6 peptides, the band in lane 2 located at the position of band 5, which represented tetra-valent avidin-biotin-peptide conjugates. Similar results were also observed in lane 3 versus lane 4, and lane 5 versus lane 6. As to tetra-valent avidin-biotin-peptide conjugates (lane 7), since they possess no vacant subunits for further biotin binding, reacting with excessive biotin-G-G-His×6 peptides did not substantially alter the band position (lane 8).

In sum, the mono-, bi-, and tri-valent avidin-biotin-peptide conjugates prepared by the step-wise process and purified with nickel affinity column retained the binding activity of the streptavidin molecule towards the biotin.

### Example 3

### Preparation of PEGs Modified with Single-Valent Avidin-Biotin-Peptide Conjugates

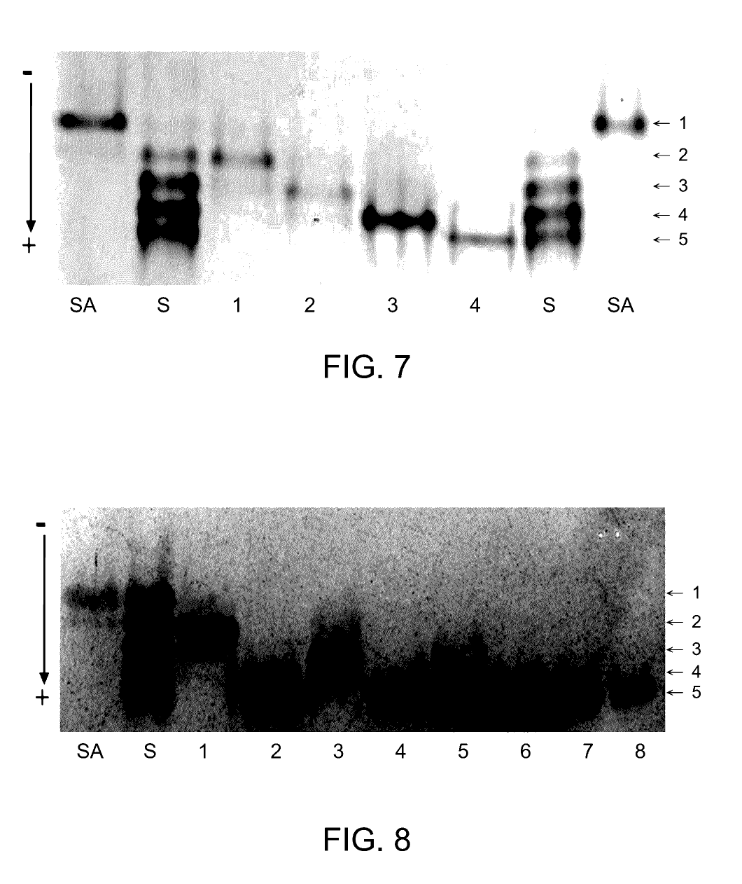

7.5 μL of 10 mM biotinylated PEGs (biotin-PEG₃ₖ-NH₂) were reacted with 7.5 μL of 2 μM mono-, bi-, tri-, or tetra-valent avidin-biotin-peptide conjugates prepared in Example 2.5, above for 5 minutes, and the products were subjected to vertical electrophoresis (8% polyacrylamide gel; 75 V, 400 mA, 10 minutes; followed by 150 V, 400 mA, 150 minutes); results were depicted in FIG. 9.

In FIG. 9, lanes SA′, 1, 3, 5, and 7 respectively contained 15 μL of 2 μM streptavidin, and mono-, bi-, tri-, and tetra-valent avidin-biotin-peptide conjugates, and the band patterns in these lane were similar to lanes 1 to 4 in FIG. 7. Specifically, in lane 1, the mono-valent avidin-biotin-peptide conjugates formed a band around band 5 labelled in FIG. 9. After reacting with biotinylated PEGs, up to three PEG molecules could bind with each mono-valent avidin-biotin-peptide conjugate, and therefore, the molecular weight of the resultant modified conjugate increased, thereby retarding the mobility thereof; for example, in lane 2, the band located around the position of band 1. Similarly, bands of bi- and tri-valent avidin-biotin-peptide conjugates were respectively formed at band 6 (in lane 3) and band 7 (in lane 5), and after reacting with biotinylated PEGs, the bands of the resultant modified conjugates moved upward to band 2 (in lane 4) and band 3 (in lane 6), respectively. On the other hand, since the tetra-valent avidin-biotin-peptide conjugates possessed no vacant subunits for biotin binding, both bands of tetra-valent avidin-biotin-peptide conjugates, before (lane 7) and after (lane 8) reacting with biotinylated PEGs, were located at band 8, as labelled in FIG. 9.

Further, in lane SA′ of FIG. 9, a streptavidin molecule had up to four PEG molecules conjugated therewith. By comparing results of lanes SA′, 2, 4, and 6, it was observed that the bands in each of the above-mentioned lanes were arranged in descending order from top to bottom, which coincided with the descending molecular sizes of the resultant conjugates in each of the above-mentioned lane.

Results of this example established that the mono-, bi-, and tri-valent avidin-biotin-peptide conjugates prepared by the method according to embodiments of the present invention were, respectively, capable of binding with three, two, or one polymeric molecule(s), such as the 3 kDa PEG molecule used herein.

### Example 4

### Preparation of Nucleotides Modified with Single-Valent Avidin-Biotin-Peptide Conjugates

4.1. Preparation of Biotinylated Nucleotides

48 kb Lambda DNA (500 μg/mL; Catalog No.N3011S, from QIAGEN) was used as the template DAN. Forward and reverse primers (synthesized by MDBio, Taipei, Taiwan) for the amplification of nucleotide fragments of various lengths were summarized in Table 1, in which the 5′-end of each reverse primer was biotinylated.

TABLE 1

| Nucleotide | | | SEQ ID |
|---|---|---|---|
| Fragment | Primer | Sequence | No. |
| 150 bp | Forward | 5′-GCTGG AGTGA | 3 |
| | | GTGGG AAGAG-3′ | |
| | Reverse | 5′-GCTGG AGTGA | 4 |
| | | GTGGG AAGAG-3′ | |
| 500 bp | Forward | 5′-ACAGC ACGGA | 5 |
| | | ACGGG TGAAG-3′ | |
| | Reverse | 5′-ACAGC ACGGA | 6 |
| | | ACGGG TGAAG-3′ | |
| 850 bp | Forward | 5′-CCAGC TGTCT | 7 |
| | | GCACA GGAGA-3′ | |
| | Reverse | 5′-CCAGC TGTCT | 8 |
| | | GCACA GGAGA-3′ | |

Chemicals for polymerase chain reaction (PCR) were prepared from the commercial PCR kit (Catalog No.PT-TMM228, from Protech). PCR solution was prepared by mixing 2 μL of 100 nM Lambda DNA, 2.5 μL of 5 μM forward primer, 2.5 μL of 5 μM reverse primer, 1 μl of 2 U/μL Taq DNA polymerase, 5 μL of 10×PCR buffer, and 3 μL of dNTPs mixture (2.5 mM each) in a microcentrifuge tube with double distilled water to a total volume of 50 μL. The PCR solution was pre-heated to 95° C. for 2 minutes, and thermocycling conditions for the PCR included, 35 cycles of denaturing at 95° C. for 15 seconds, annealing at 55° C. for 15 seconds, and elongation at 72° C. for 30 seconds, followed by a final extension at 72° C. for 5 minutes. The PCR products were subjected to agarose gel electrophoresis (2% gel; 0.5×TAE buffer (4.84 Tris (Sigma), 1.14 mL of acetic acid, and 0.37 g ethylenediamine-tetraacetic acid (EDTA, Sigma) were mixed with double distilled water to a total volume of 1 Liter; filtered through 0.22 μm filter and stored at room temperature); 100 V; 50 minutes), and the biotinylated DNA fragments were extracted from the cutting band of the agarose gel using QlAquick Gel Extraction Kit (QIAGEN).

4.2. Preparation of Nucleotide/Single-Valent Avidin-Biotin-Peptide Conjugates

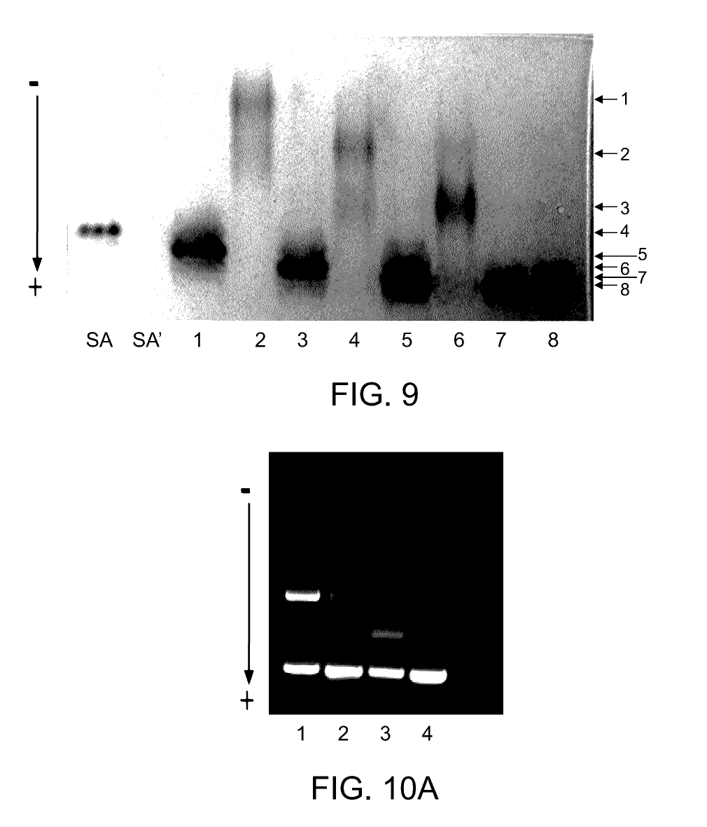

15 μL of 600 μg/μL nucleotide fragments (prepared in Example 4.1, above) were mixed with 15 μL of 1 μM mono-, bi-, tri-, or tetra-valent avidin-biotin-peptide conjugates (from Example 2.5, above). After 120 minutes of reaction, each sample was subjected to agarose electrophoresis (4% for 150-bp DNA fragments, and 2% for 500- and 850-bp DNA fragments; 0.5×TAE buffer; 10 V; 50 minutes). The agarose gel was stained with 0.5 μg/mL ethidium bromide (EtBr) in the dark and the DNA fragments were then imagined using a UV imaging system (UVITOC; from UVITEC), and the results are depicted in FIGS. 10A to 10C. Through FIGS. 10A to 10C, lane SA contained biotinylated DNA fragments reacted with streptavidin, lane C was loaded with biotinylated DNA fragments only, whereas lanes 1 to 4 respectively contained biotinylated DNA fragments reacted with mono-, bi-, tri-, and tetra-valent avidin-biotin-peptide conjugates.

The results, as illustrated in FIGS. 10A to 10C, indicated that tetra-valent avidin-biotin-peptide conjugates, as expected, were not active for binding with any biotinylated DAN fragments (lane 4), whereas mono-, bi-, or tri-valent avidin-biotin-peptide conjugates were capable of conjugating with one or more biotinylated DNA fragments (lanes 1 to 3).

### Example 5

### Preparation of Gold Nanoparticles Modified with Single-Valent Avidin-Biotin-Peptide Conjugates

5.1 Synthesis of 6 nm-AuNPs-PEG-Biotin

Gold nanoparticles having an average diameter of 6 nm (6 nm-AuNPs) were prepared by the following method. First, didodecyldimethylammonium bromide (DAAB; Fluka 36785) solution (100 mM in toluene (Tedia TS-2132-001)), AuCl₃ solution (Aldrich 379948; 7.5 mg/mL in DAAB), decanioc acid solution (Sigma L4250; 17.2 mg/mL in toluene), tetrabutylammonium borohydride (TBAB; Fluka 86855) solution (25 mg/mL in DDAB), and trioctylphosphine (TOP) solution (0.1 M in toluene) were respectively prepared. Thereafter, 2.5 mL of decanioc acid solution and 1 mL of TBAB solution were mixed using the Corning stirrer/hotplate; after the reaction mixture was stabilized, 0.8 mL of AuCl₃ solution was injected with vigorous stirring. The solution color changed to deep red within one minute of mixing, indicating particle formation. Particles were stable for more than 24 hours.

The 6 nm-AuNPs were coated with amphiphilic polymers, which were synthesized by grafting the hydrophobic alkylamine onto the hydrophilic poly(maleic anhydride) backbone through spontaneous amide linkage. In general, 3.084 g (20 mmol monomer) of poly(isobutylene-alt-maleic anhydride) (Sigma 531278) were placed in a round flask. 15 mmol dodecylamine (Sigma 325163) dissolved in 100 mL of anhydrous tetrahydrofuran (THF; Aldrich 186562) were quickly injected and vigorously mixed with the polymer powders. The cloudy mixture was sonicated for some seconds and then kept at 60° C. under vigorous stirring, and the solution became transparent in about 1 minute. The reaction mixture was concentrated until the volume was reduced to about one third of its original volume by a rotavapor system (LABOROTA 4001) under a reduced pressure (p=500 to 300 mbar). The concentrated solution was further incubated overnight at 60° C. under stirring. The solvent was then evaporated until the polymer was completely dry (pale yellow solid). Finally, the resulting amphiphilic polymer was re-dissolved in anhydrous chloroform (Tedia CS-1332) and adjusted to a final volume of 25 mL, yielding a calculated concentration of monomer units of 0.8 M.

For polymer coating, 5 mL chloroform, 6 nm-AuNPs (13.76 mg), and 0.8 M amphiphilic polymer (300 μL) were sequentially added into a round flask. Then, the reaction mixture was concentrated using a rotavapor system (LABOROTA 4001) under a reduced pressure (starting pressure 200 mbar; reduced to 1 mbar at a rate of 50 mbar/5 minutes) until the reaction mixture was completely dry. The resulting product was re-dissolved in 0.1 N NaOH, incubated at 80° C. water bath for 5 minutes, and filtered with a 0.22 μm filter. The filtrate was centrifuged (40,000 rpm, 20 minutes, 4° C.) for three times; after each centrifugation, the supernatant was collected, and 0.1N NaOH was added into the centrifuge tube. The collected supernatants were concentrated with 100 kDa molecular sieve, and the resultant polymer—coated 6 nm—AuNPs were stored in sodium borate (SBB) buffer (; 19.07 g sodium borate (Na₂B₄O₇, from J. T. Baker) and 3.07 g boric acid (H₃BO₃, from Sigma Aldrich) were mixed with double distilled water to a total volume of 1 L; adjusted to pH 9, filtered with 0.22 μm filter; stored at room temperature).

To produce gold nanoparticles modified with polyethylene glycol (PEG), 10 μL of 8 mM N-(3-Dimethylaminopropyl)-N′-ethylcarbodiimide (EDC) was added into the mixture of 10 μL of 2 μM polymer-coated 6 nm-AuNPs and 10 μL of 0.25 mM biotinylated PEG (Biotin-PEG₍₅₀₀₀₎-NH₂), and the reaction mixture was allowed to react for 2 hours. The products were separated by vertical electrophoresis (2% agarose gel; 0.5×TBE buffer (10.8 g Tris, 5.5 g boric acid, and 0.244 g EDTA were mixed with double distilled water to a total volume of 2 L; filtered through 0.22 μm filter and stored at room temperature); 75 V; 400 mA, 90 minutes), followed by dialysis (2% agarose gel; 0.5×TBE buffer; 100 V; 400 mA, 20 minutes). After sieving with 100 kDa molecular sieve, the sample was purified by vertical electrophoresis (2% agarose gel; 0.5×TBE buffer; 75 V; 400 mA, 90 minutes) and dialysis (2% agarose gel; 0.5×TBE buffer; 100 V; 400 mA, 20 minutes). The resulting product, Biotin-PEG₅₀₀₀₎@AuNPs_{(6 nm)} (or 6 nm-AuNPs-PEG-Biotin), was filtered using a 0.22 μm filter, sieved with 100 kDa molecular sieve, and then stored in SBB buffer.

5.2 Preparation of 6 nm-AuNPs-PEG-Biotin/Single-Valent Avidin-Biotin-Peptide Conjugates

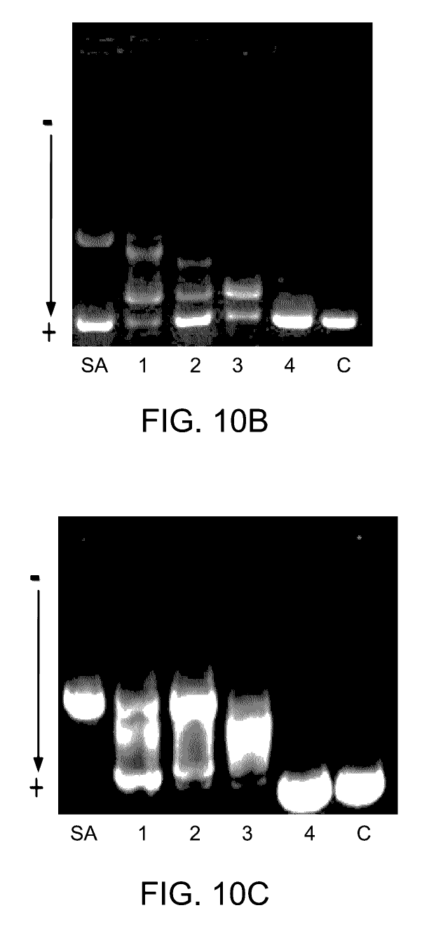

10 μL of 1.5 μM 6 nm-AuNPs-PEG-Biotin (from Example 5.1, above) were reacted with 10 μL of streptavidin solutions ranging from 0.25 to 10 μM for two hours. Thereafter, 2 μL of loading buffer was injected into each sample, which was then subjected to electrophoresis (3% agarose gel; 0.5×TBE buffer; 75 V; 400 mA, 90 minutes); results are illustrated in FIG. 11.

The electrophoresis results provided in FIG. 11 indicated that when the molar ratio between 6 nm-AuNPs-PEG-Biotin and streptavidin was 1.5:1 (lane 4), each streptavidin molecule was conjugated with one or two 6 nm-AuNPs-PEG-Biotin molecules. When the molar ratio increased to 3:1 (lane 5), most streptavidin molecules were conjugated with two 6 nm-AuNPs-PEG-Biotin molecules, and some streptavidin molecules were conjugated with three 6 nm-AuNPs-PEG-Biotin molecules. As the molar ratio further increased to 6:1 (lane 6), each streptavidin molecule was conjugated with three or four 6 nm-AuNPs-PEG-Biotin molecules.

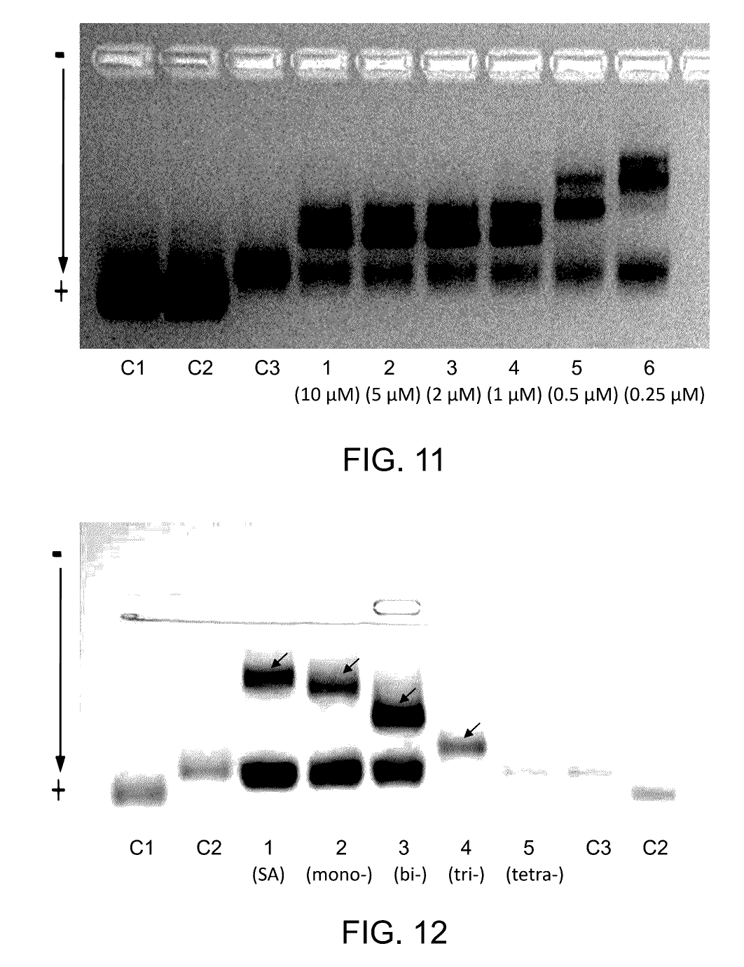

Next, 10 μL of 2 μM 6 nm-AuNPs-PEG-Biotin were reacted with 10 μL of streptavidin solution (0.5 μM) or mono-(0.1 μM), bi-(0.2 μM), tri-(0.5 μM), or tetra-valent avidin-biotin-peptide conjugates (0.5 μM) prepared in Example 2.5, above. After reacting for 2 hours, 3 μL of loading buffer was injected into each sample, which was then subjected to electrophoresis (4% agarose gel; 0.5×TBE buffer; 75 V; 400 mA, 60 minutes). The electrophoresis results were provided in FIG. 12.

The photograph in FIG. 12 established that the vacant biotin-binding site of the present single-valent avidin-biotin-peptide conjugate retained the binding activity toward the biotin moiety of the biotinylated, amphiphilic gold nanoparticles (6 nm-AuNPs-PEG-Biotin). Specifically, the upper band (indicated by arrow head) in lane 2 contained 6 nm-AuNPs-PEG-Biotin modified with mono-valent avidin-biotin-peptide conjugates, in which three 6 nm-AuNPs-PEG-Biotin molecules were conjugated onto one mono-valent avidin-biotin-peptide conjugate. Similarly, the upper band (indicated by arrow head) in lane 3 contained modified nanoparticles in which two 6 nm-AuNPs-PEG-Biotin molecules were conjugated onto one bi-valent avidin-biotin-peptide conjugate. As to lane 4, each modified nanoparticle comprises one 6 nm-AuNPs-PEG-Biotin molecule conjugated with one tri-valent avidin-biotin-peptide conjugate.

Gel strips containing bands (indicated by arrow heads) from lanes 1 to 4 of FIG. 12 were excised and eluted by electrophoresis into the dialysis bag (4% agarose gel; 75 V; 60 minutes). 10 μL of the purified product was loaded onto a mica strip (1 cm²) and let stand for about 30 minutes until completely dried, before being subjected to Atomic force microscope (AFM, Nano WizardRII BioAFM) analysis.

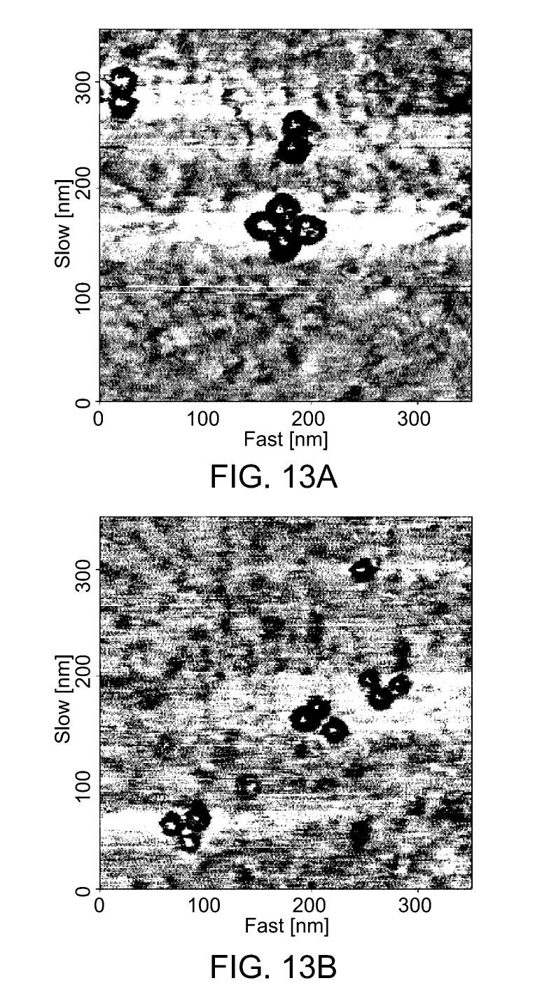

The Phase images captured by AMF were provided in FIGS. 13A to 13D. In FIG. 13A, the sample was taken from the indicated band of lane in FIG. 12, and the image showed that four 6 nm-AuNPs-PEG-Biotin molecules were conjugated onto a streptavidin molecule. The AMF images in FIGS. 13B to 13C confirmed that the present mono-, bi-, and tri-valent avidin-biotin-peptide conjugates were capable of binging with three, two, and one 6 nm-AuNPs-PEG-Biotin molecule(s) per conjugate.

5.3. Synthesis of 15 nm-AuNPs-PEG-Biotin

Gold nanoparticles having an average diameter of 15 nm (15 nm-AuNPs) were prepared as follows. First, 50 mg chloroauric acid (HAuCl₄) and 4.1 mL of oleylamine (Acrosorganics) were added in 7.5 mL of toluene pre-heated in oil bath at 110° C. The solution was stirred continuously for 2 hours. The solution color of deep red indicated the formation of the particles.

The 15 nm-AuNPs were coated with amphiphilic polymer, biotinylated, and purified as described in Example 5.1, above, except that biotin-PEG₁₀ₖ-NH₂ was used for the biotinylation of 15 nm-AuNPs. The resulting product, Biotin-PEG_{(10K)}@AuNPs_{(15 nm)} (or 15 nm-AuNPs-PEG-Biotin), was filtered using a 0.22 μm filter, sieved with 100 kDa molecular sieve, and then stored in SBB buffer.

5.4. Preparation of 6 nm-AuNPs/15 nm-AuNPs-PEG-Biotin/Single-Valent Avidin-Biotin-Peptide Conjugates

In this example, polymer-coated 6 nm-AuNPs (prepared in Example 5.1, above) were first reacted with tri-valent avidin-biotin-peptide conjugates (prepared in Example 2.5, above) or streptavidin; then the resultant conjugates were reacted with 15 nm-AuNPs-PEG-Biotin (prepared in Example 5.3, above) to produce 6 nm-AuNPs/15 nm-AuNPs-PEG-biotin/single-valent avidin-biotin-peptide conjugates.

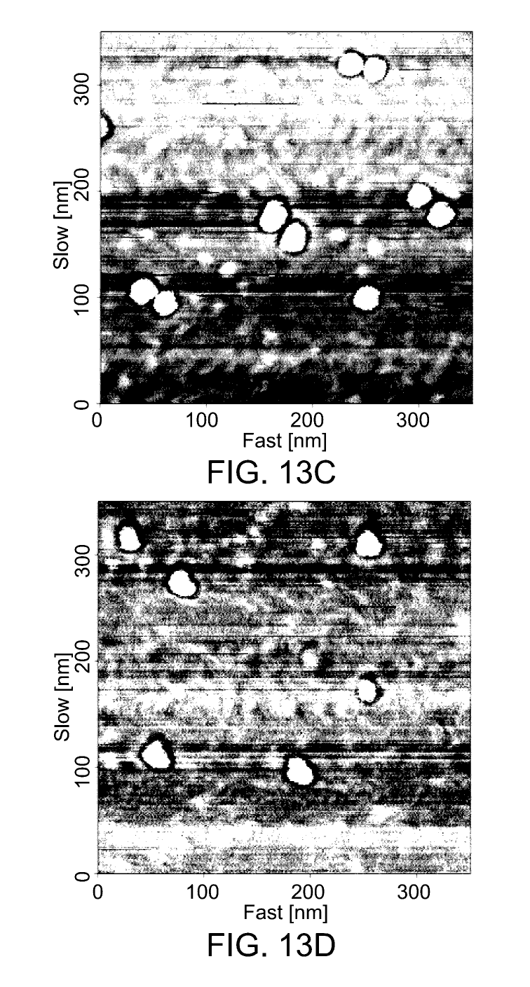

Specifically, 200 μL of 2 μM polymer-coated 6 nm-AuNPs and 200 μL of 16 mM N-(3-dimethylaminopropyl)-N′-ethylcarbodiimide (EDC) were mixed with 200 μL of 5 μM streptavidin solution or 200 μL of 5 μM tri-valent avidin-biotin-peptide conjugates. The reaction mixture was allowed to react for 2 hours so that amine group of the streptavidin reacted with the carboxyl group of the amphiphilic polymer under the activation of EDC to form an amide bonding, thereby producing 6 nm-AuNP/streptavidin conjugates or 6 nm-AuNPs/tri-valent avidin-biotin-peptide conjugates. Thereafter, 15 μL of 30 nM biotinylated 15 nm-AuNPs were mixed with 15 μL of 6 nm-AuNPs/tri-valent avidin-biotin-peptide conjugates or 6 nm-AuNP/streptavidin conjugates of various concentrations in the range of 0.025 μM to 0.8 μM (2-fold serial dilution). After reacting overnight, the products were subjected to electrophoresis (4% agarose gel; 75 V; 400 mA; 30 minutes) and the results were provided in FIG. 14.

Referring to lanes 1 to 6 in FIG. 14, the biotinylated 15 nm-AuNPs were reacted with 6 nm-AuNPs/tri-valent avidin-biotin-peptide conjugates. The lowest band (indicated by arrow head) in lane 1 located at the 10o same height with respect to the band in lane C2 (1 μM 6 nm-AuNP/tri-valent avidin-biotin-peptide conjugates), indicating that the indicated band in lane 1 also contained 6 nm-AuNP/tri-valent avidin-biotin-peptide conjugates. In lane 2, the band indicated by arrow head located at a higher position, as compared with the lowest band in lane 1, suggesting the presence of 6 nm-AuNPs/15 nm-AuNPs-PEG-biotin/single-valent avidin-biotin-peptide conjugates in the indicated band of lane 2. However, as the concentration of polymer-coated 6 nm-AuNPs decreased, fewer 6 nm-AuNPs/15 nm-AuNPs-PEG-biotin/single-valent avidin-biotin-peptide conjugates were formed (see, lanes 1 to 6 with descending concentrations).

In lanes 7 to 12, the biotinylated 15 nm-AuNPs were reacted with 6 nm-AuNP/streptavidin conjugates. The lowest band (indicated by arrow head) in lane 7 contained molecules of the smallest size (i.e., the 6 nm-AuNP/streptavidin conjugates). The band indicated by arrow head in lane 8 contained the conjugates of one 15 nm-AuNP and one 6 nm-AuNP/streptavidin conjugate, whereas the band indicated by arrow head in lane 9 contained the conjugates of two 15 nm-AuNPs and one 6 nm-AuNP/streptavidin conjugate.

Gel strips containing bands (indicated by arrow heads) from lanes 2 and 9 of FIG. 14 were excised and eluted by electrophoresis into the dialysis bag (4% agarose gel; 75 V; 60 minutes). The purified sample was loaded on the TEM copper grid; after 10 seconds, excess sample was removed using bottom filter paper. Before imaging with the Hitachi H-7100 Transmission Electron Microscope, the sample was put in the drying oven for more than 4 hours until completely dry.

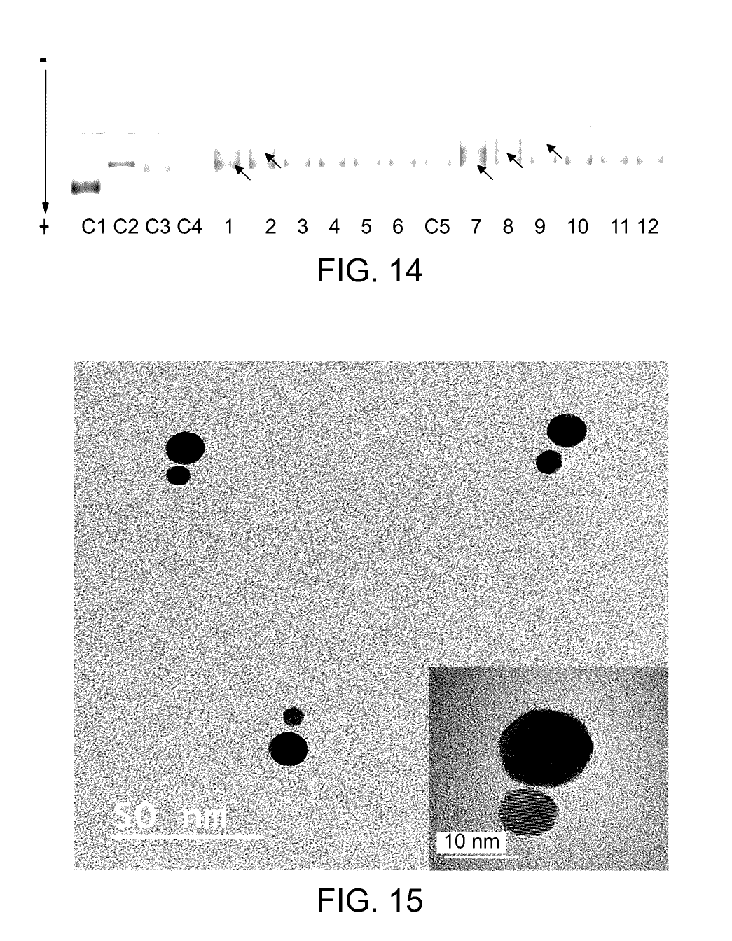

As could be appreciated, each 6 nm-AuNPs/tri-valent avidin-biotin-peptide conjugates possessed only a single vacant biotin-binding site, and hence, only one biotinylated 15-nm AuNP could conjugated with one 6 nm-AuNPs/tri-valent avidin-biotin-peptide conjugate, as is evidenced by the TEM image of FIG. 15, in which conjugates having two nanoparticles of different sizes were observed.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method for preparing single-valent, modified avidin-like molecules, comprising the steps of,
(a) adding a plurality of biotinylated peptides into a plurality of avidin-like molecules in a molar ratio between 1:4 to 16:1, wherein 1% to 10% of the total number of the plurality of avidin-like molecules are added into the plurality of avidin-like molecules per minute, so as to form a plurality of avidin-biotin-peptide conjugates having different valence states, wherein each of the plurality of biotinylated peptides comprises a His-tag sequence, and the avidin-like molecule is selected from the group consisting of, avidin, streptavidin, NeutrAvidin, and CaptAvidin; and
(b) separating the plurality of avidin-biotin-peptide conjugates of the step (a) by use of a nickel affinity column eluted with an imidazole gradient to produce a first eluate consisting essentially of a plurality of single-valent avidin-biotin-peptide conjugates respectively having a first valence.

2-3. (canceled)

4. The method of claim 1, wherein
the sequence of the biotinylated peptide is the amino acid sequence of SEQ ID No. 1, and the C-terminus of the amino acid sequence is biotinylated; or
the sequence of the biotinylated peptide is the amino acid sequence of SEQ ID No. 2, and the N-terminus of the amino acid sequence is biotinylated.

5. The method of claim 1, wherein the biotinylated peptide further comprises an amino acid sequence of a cell-targeting sequence or a cell-penetrating sequence, locating at the N-terminus or C-terminus of the His-tag sequence.

6. The method of claim 1, wherein the imidazole gradient is 5 mM to 1,500 mM imidazole.

7. A method of preparing a plurality of functional conjugates, wherein each functional conjugates comprises a single-valent, modified avidin-like molecules and one or more first functional molecules, and the method comprises the steps of,
(a) adding a plurality of biotinylated peptides into a plurality of avidin-like molecules in a molar ratio between 1:4 to 16:1, wherein 1% to 10% of the total number of the plurality of avidin-like molecules are added into the plurality of avidin-like molecules per minute, so as to form a plurality of avidin-biotin-peptide conjugates having different valence states, wherein each of the plurality of biotinylated peptides comprises a His-tag sequence, and the avidin-like molecule is selected from the group consisting of, avidin, streptavidin, NeutrAvidin, and CaptAvidin;
(b) separating the plurality of avidin-biotin-peptide conjugates of the step (a) by use of a first nickel affinity column eluted with an imidazole gradient to produce a first eluate consisting essentially of a plurality of first single-valent avidin-biotin-peptide conjugates respectively having a first valence; and
(c) mixing a plurality of first functional molecules with the plurality of first single-valent avidin-biotin-peptide conjugates to form a plurality of first avidin-biotin-peptide/first functional molecule conjugates.

8. The method of claim 7, wherein each of the plurality of first functional molecules comprises a first functional moiety selected from the group consisting of, nucleotides, peptides, antibodies, antigens, nanoparticles, quantum dots, fluorescent materials, magnetic materials, and therapeutics.

9. The method of claim 7, wherein each of the plurality of the first functional molecules comprises a reaction group capable of reacting with the avidin-like molecules, thereby binding with the avidin-like molecules.

10. The method of claim 9, further comprising the step of,
(d) mixing a plurality of second functional molecules with the plurality of first avidin-biotin-peptide/first functional molecule conjugates so as to form a plurality of first avidin-biotin-peptide/first functional molecule/second functional molecule conjugates, wherein each of the plurality of the second functional molecules comprises a second avidin-binding moiety or a second His-tag-binding moiety.

11. The method of claim 10, wherein each of the plurality of second functional molecules further comprises a second functional moiety covalently bound to the second avidin-binding moiety or the second His-tag-binding moiety, wherein the second functional moiety is selected from the group consisting of, nucleotides, peptides, antibodies, antigens, nanoparticles, quantum dots, fluorescent materials, magnetic materials, and therapeutics.

12. The method of claim 7, wherein each of the plurality of the first functional molecules comprises an avidin-binding moiety or a His-tag-binding moiety, and in the step (c), the molar ratio between the plurality of first functional molecules and the plurality of single-valent avidin-biotin-peptide conjugates 1:4 to 16:1.

13. The method of claim 12, wherein in the step (c), the number of the plurality of first functional molecules added into the plurality of first single-valent avidin-biotin-peptide conjugates per minute is 1% to 10% of the total number of the plurality of first single-valent avidin-biotin-peptide conjugates.

14. (canceled)

15. The method of claim 7, wherein in the step (b), the at least one eluate further comprises a second eluate consisting essentially of a plurality of second single-valent avidin-biotin-peptide conjugates respectively having a second valence that is different from the first valence, and the method further comprises the step of,
(e) mixing a plurality of second functional molecules with the plurality of second single-valent avidin-biotin-peptide conjugates to form a plurality of second avidin-biotin-peptide/second functional molecule conjugates.

16. The method of claim 15, wherein each of the plurality of second functional molecules comprises a second functional moiety selected from the group consisting of, nucleotides, peptides, antibodies, antigens, nanoparticles, quantum dots, fluorescent materials, magnetic materials, and therapeutics.

17. (canceled)

18. The method of claim 7, wherein
the sequence of the biotinylated peptide is the amino acid sequence of SEQ ID No. 1, and the C-terminus of the amino acid sequence is biotinylated; or
the sequence of the biotinylated peptide is the amino acid sequence of SEQ ID No. 2, and the N-terminus of the amino acid sequence is biotinylated.

19. The method of claim 7, wherein the biotinylated peptide further comprises an amino acid sequence of a cell-targeting sequence or a cell-penetrating sequence, locating at the N-terminus or C-terminus of the His-tag sequence.

20. The method of claim 7, wherein the imidazole gradient is 5 mM to 1,500 mM imidazole.
